(19) United States
(12) Patent Application Publication
(10) Pub. No.: US 2007/0100339 A1
(43) Pub. Date: May 3, 2007

(54) Vertebral osteosynthesis equipment

(75) Inventors: Jean-Luc Clement, La Colle Sur Loup (FR); Vincent Fiere, Lyon (FR); Jean Taylor, Cannes (FR); Yves Adam, Authie (FR); Bernard Villaret, Croix-Chapeau (FR)

Correspondence Address: YOUNG & THOMPSON, 745 SOUTH 23RD STREET, 2ND FLOOR, ARLINGTON, VA, 22202, US

(73) Assignee: MEDICREA TECHNOLOGIES, LA ROCHELLE (FR)

(21) Appl. No.: 10/562,203

(22) PCT Filed: Jun. 24, 2004
(86) PCT No.: PCT/IB04/02458

§ 371 Date: Apr. 10, 2006

Related U.S. Application Data
(60) Provisional application No. 60/490,520, filed on Jul. 29, 2003.

(30) Foreign Application Priority Data
Jun. 27, 2003 (FR) 0307777

(51) Int. Cl. A61B 17/70

(52) U.S. Cl. 606/61

(57) ABSTRACT

Equipment includes bony anchoring members one or two linking rods to be connected to the anchoring members and be attached to the vertebrae by dint thereof, parts for connecting the linking rod(s) to the anchoring members, and clamping elements for locking the linking rod(s) in the connecting parts; at least one anchoring member includes one proximal stud articulated with respect to a body for gripping a vertebra. The proximal stud exhibits an articulation head in the form of a spherical cap; and the anchoring member body includes a cavity for accommodating the articulation head and a wall surrounding this cavity, this wall being crimped around the articulation head and shaped to provide a proximal form, at least partially hemispherical.

This patent application claims the priorities to:
FR 03 07777, filed on Jun. 27, 2003;
U.S. Provisional application No. 60/490,520, filed on Jul. 29, 2003.

The present invention relates to vertebral osteosynthesis equipment and a manufacturing process of a bony anchoring member included in such equipment.

A vertebral osteosynthesis equipment generally includes bony anchoring members, such as pedicular screws or lamar hooks, one or two linking rods, intended to be connected to these anchoring members and to be attached to the vertebrae by dint thereof, and parts for connecting this(these) linking rod(s) to these anchoring members. The equipment may also comprise length-adjustable crossbeams, which link transversally two parallel linking rods in order to hold said rods with respect to one another.

In an existing type of equipment, each anchoring member comprises a proximal threaded stud whereon a nut may be screwed, and each connecting part comprises a rounded section intended for surrounding a linking rod and two parallel drilled wings. These wings are intended for engaging onto said proximal threaded stud and for being clamped, by means of that nut, against a bearing surface provided on the anchoring member, said operation enabling to clamp said rounded section around the linking rod and thereby ensuring longitudinal immobilisation of this rod with respect to the anchoring member. The anchoring members may be of “monoaxial” type, i.e. comprise a proximal threaded stud integral with of the portion of the anchoring member intended for gripping the bone, or may be of “polyaxial” type, i.e. comprise a proximal threaded stud articulated with respect to that portion intended for gripping the bone.

A polyaxial anchoring member known comprises, for the purpose of articulation of said proximal stud, a spherical head integral with this stud, received in a corresponding spherical cavity. In several types of existing anchoring members, this cavity is formed by providing a spherical semi-cavity in the proximal zone of the anchoring member and a portion of spherical cavity in the internal zone of a shouldered nut which is screwed on this proximal zone, this nut and this proximal zone locking said spherical head therebetween.

The shortcoming of the anchoring members known is that, after implantation, they protrude high above the bony zone whereon they are placed. These significant heights, if they are admissible for treating certain locations of a vertebral spine, in particular the lumbar vertebrae or the lumbar sacral articulation, may prove disturbing, let alone unacceptable, for treating other segments of a vertebral spine, in particular dorsal vertebrae, or for implanting particular equipment such as equipment placed laterally on the vertebrae to be treated.

A polyaxial anchoring member known comprises moreover a proximal gripping portion enabling of hold said member when clamping the nut. This proximal gripping portion, notably in the form of a hexagonal zone intended for co-operating with a corresponding holding key on known equipment, contributes to confer significant height to the anchoring member.

The purpose of the present invention is to remedy the shortcomings resulting from the height of a polyaxial anchoring member above the bony zone whereon this anchoring member is placed.

The vertebral osteosynthesis equipment affected comprises bony anchoring members, such as pedicular screws and/or lamar hooks, one or two linking rods, intended to be connected to these anchoring members and to be attached to the vertebrae by dint thereof, parts for connecting this(these) linking rod(s) to these anchoring members, and clamping means, such as nuts, for locking the linking rod(s) in said connecting parts; at least one of said bony anchoring members is of the “polyaxial” type, i.e. comprises one proximal stud articulated with respect to a body intended for gripping a vertebra.

According to the invention,
the proximal stud of said polyaxial anchoring member exhibits an articulation head in the form of a spherical cap; and
said body of this anchoring member comprises a cavity intended for accommodating this articulation head and a wall surrounding this cavity, this wall being crimped around said articulation head and shaped in order to provide a proximal form, at least partially hemispherical.

The anchoring member according to the invention does not comprise therefore any spherical head no shouldered nut for capturing this spherical head but an articulation head in the form of a spherical cap retained in an articulation cavity by a wall crimped around said cavity.

The height of the portion of the anchoring member according to the invention which protrudes from a vertebra after implantation is, consequently, quite notably reduced with respect to the height of the anchoring members according to prior techniques.

The method according to the invention comprises the steps consisting in:
providing, on the part intended for the proximal stud of said polyaxial anchoring member, an articulation head in the form of a spherical cap;
providing a cavity in the proximal zone of the part intended for said body of said anchoring member polyaxial, and, around this cavity, a wall which may be crimped;
engaging said articulation head into said cavity, and
crimping said wall around said articulation head so that this wall exhibits a proximal form at least partially hemispherical.

Said body comprises advantageously a proximal gripping portion enabling to hold said body when tightening said clamping means, this proximal gripping portion being formed of a collar exhibiting several radial notches, notably four notches at 90° to one another.

This proximal gripping portion exhibits a height which is noticeably smaller than that of a hexagonal zone according to the prior technique, and thus contributes to limiting the height of the anchoring member.

Preferably, the equipment according to the invention comprises at least one connecting part exhibiting a rounded section intended for surrounding a linking rod and two parallel drilled wings, these wings being intended for engaging onto said proximal stud and for being clamped, using said clamping means, against a bearing surface contained in said polyaxial anchoring member; the distal wing of this connecting part exhibits moreover a distal cavity in the form of a spherical cap, of greater diameter than that of said crimped wall, the connecting part being intended for resting against this crimped wall at of this cavity.

This cavity, its diameter being greater than that of said crimped wall, enables to lock said proximal stud in an angular position with respect to the body of the anchoring member.

Advantageously, said clamping means is a nut and the proximal branch of this connecting part comprises a proximal cavity wherein a corresponding zone exhibited by this nut is intended for engaging.

After assembly, the nut is thus partially effaced in this cavity, which contributes further to reducing the height of the equipment above a vertebra, after implantation.

Preferably, this cavity, and said corresponding zone of the nut, are conical in shape, in order to increase the bearing surface of the nut against the connecting part.

The proximal stud and said connecting part may comprise means enabling to immobilise the proximal stud in rotation when the connecting part is engaged on this proximal stud. These means may in particular be at least one flat surface provided on the proximal stud and at least one flat surface provided on the connecting part, whereas these flats surfaces are immediately close to one another when the connecting part is engaged on the proximal stud.

The invention will be better understood, and other characteristics and advantages thereof will appear, with reference to the appended schematic drawing, representing, for non-limiting exemplification purposes, a preferred embodiment of parts included in the equipment affected.

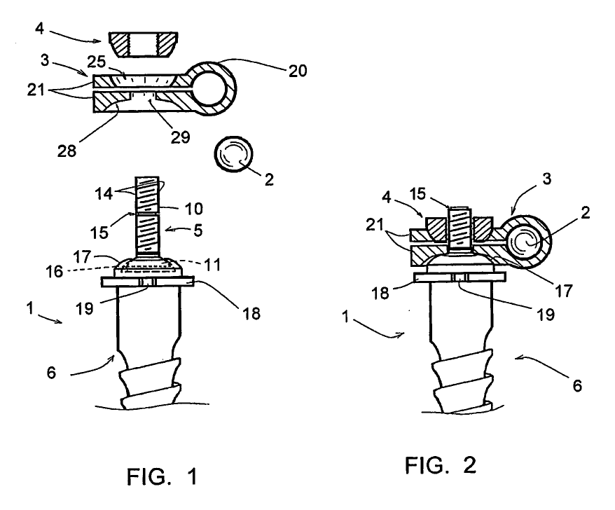

FIG. 1 is a partial view, before assembly, of a polyaxial pedicular screw, of a linking rod, represented as seen from its end, and, as a cross sectional view, of a connecting part and of a nut enabling to assemble a linking rod to this screw; and

FIG. 2 is a view of these parts similar to FIG. 1, after assembly.

FIG. 1 represents a polyaxial pedicular screw 1, a linking rod 2 for connecting several of these screws 1, a part 3 for connecting this rod 2 to one of these screws 1 and a nut 4 enabling to assemble the linking rod 2 to this screw 1.

The screw 1 comprises a proximal threaded stud 5 and a threaded distal screw body 6. The stud 5 is intended for receiving the part 3 engaged thereon and the nut 4 screwed thereon while the body 6 is intended for insertion into the pedicula of a vertebra.

The stud 5 exhibits a threaded cylindrical portion 10 and an enlarged distal head 11.

The portion 10 exhibits a zone 15 of reduced diameter, enabling to break its proximal portion after placing and clamping the nut 4, as appears by comparison of FIGS. 1 and 2.

This portion 10 also comprises two lateral flat surfaces 14.

The head 11 exhibits a diameter of the order of double the diameter of the portion 10 and looks like a spherical cap. This head 11 is intended for engaging in a proximal cavity 16 delineated by the proximal zone of the body 6 and for retention in this cavity 16 by crimping a proximal wall 17 exhibited by this body 6 around the cavity 16. After crimping, the wall 17 is shaped in order to have a proximal hemispherical form. As shown on FIG. 1, the dimensions of the cavity 16 and of the aperture delineated by the wall 17 after crimping to let through the stud 5 are such that a multidirectional backlash of this stud 5 with respect to the body 6 is possible.

The body 6 also comprises a proximal collar 18, intended for abutting against the pedicula of the vertebra treated. This collar 18 exhibits several radial notches 19, notably four notches at 90° to one another, for holding the body 6 in rotation when clamping the nut 4.

The linking rod 2 is cylindrical and exhibits such rigidity as to hold several vertebrae with respect to one another. This rod 2 is however deformable in order to be shaped relative to the correction of the rachis to be performed.

The connecting part 3 comprises a rounded section 20 intended for surrounding the linking rod 2 and two parallel lateral wings 21 drilled with holes for engaging the part 3 on the stud 5. These wings 21 are distant mutually so that, in a distant position, the rod 2 may be inserted and may slide in the portion 20, and that, in a close position provided by the clamping of the nut 4, they clamp the portion 20 around the rod 2, immobilising the latter with respect to the part 3.

As shown on Figures, the proximal wing 21 exhibits a proximal pan 25 whereof the shape is suitable for the nut 4 to rest on, the latter exhibiting a corresponding conical zone. The distal wing 21 exhibits, for its own part, a distal cavity 28 in the form of a spherical cap, whereof the diameter is greater than that of the wall 17, and a hole 29. This hole 29 comprises two flat surfaces whereof the distance is slightly greater than that between both flat surfaces 14, so that when the stud 5 is engaged in this hole, the stud 5 is immobilised in rotation with respect to the part 3.

In practice, the number of screws 1 necessary to the treatment to be performed is placed in the pediculae of the vertebrae affected, then the connecting parts 3, with the rod 2 engaged in the portions 20, are placed on the studs 5. The nuts 4 are then clamped to immobilise the rod 2 with respect to the parts 3 and the proximal portions of the studs 5 are cut off.

As shown by the foregoing, the invention provides a vertebral osteosynthesis equipment whereof the anchoring members only protrude marginally beyond the vertebrae wherein they are implanted and are, consequently, only little sensitive under the skin, or even not sensitive at all. Such equipment may thus be used for treating not only lumbar vertebrae or the lumbar sacral articulation, but also dorsal vertebrae or for implanting equipment placed laterally on the vertebrae to be treated.

It is obvious that the invention is not limited to the embodiment described above for exemplification purposes but that it extends to all the embodiments covered by the claims appended therein.

1. Vertebral osteosynthesis equipment, including bony anchoring members, such as pedicular screws (1) and/or lamar hooks, one or two linking rods (2), intended to be connected to these anchoring members and to be attached to the vertebrae by dint thereof, parts (3) for connecting this(these) linking rod(s) (2) to these anchoring members, and clamping means, such as nuts (4), for locking the linking rod(s) (2) in said connecting parts (3); at least one anchoring member is of the “polyaxial” type, i.e. comprises one proximal stud (5) articulated with respect to a body (6) intended for gripping a vertebra; equipment characterized in that
the proximal stud (5) of said polyaxial anchoring member exhibits an articulation head (11) in the form of a spherical cap; and
said body (6) of this anchoring member comprises a cavity (16) intended for accommodating this articulation head (11) and a wall (17) surrounding this cavity (16), this wall (17) being crimped around said articulation head (11) and shaped in order to provide a proximal form, at least partially hemispherical.

2. Vertebral osteosynthesis equipment according to claim 1, characterized in that said body (6) comprises a proximal gripping portion enabling to hold said body (6) when tightening said clamping means, this proximal gripping portion being formed of a collar (18) exhibiting several radial notches (19).

3. Vertebral osteosynthesis equipment according to claim 2, characterized in that said collar (18) exhibits four notches (19) at 90° to one another.

4. Vertebral osteosynthesis equipment according to claim 1, characterized in that comprises at least one connecting part (3) exhibiting a rounded section (20) intended for surrounding a linking rod (2) and two parallel drilled wings (21), these wings (21) being intended for engaging onto said proximal stud (5) and for being clamped, using said clamping means (4), against a bearing surface (17) contained in said polyaxial anchoring member; the distal wing (21) of this connecting part (3) exhibits a distal cavity (28) in the form of a spherical cap, of greater diameter than that of said crimped wall (17), the connecting part (3) being intended for resting against this crimped wall (17) at of this cavity (28).

5. Vertebral osteosynthesis equipment according to claim 4, characterized in that said clamping means is a nut (4) and the proximal branch (21) of this connecting part (3) comprises a proximal cavity (25) wherein a corresponding zone exhibited by this nut (4) is intended for engaging.

6. Vertebral osteosynthesis equipment according to claim 5, characterized in that said cavity (25) and said corresponding zone of the nut (4) are conical in shape.

7. Vertebral osteosynthesis equipment according to claim 1, characterized in that the stud (5) exhibits a zone (15) of reduced diameter, enabling to break its proximal portion after placing and clamping the nut (4).

8. Vertebral osteosynthesis equipment according to claim 1, characterized in that the proximal stud (5) and said connecting part (3) comprise means (14, 29) enabling to immobilise the proximal stud (5) in rotation when the connecting part (3) is engaged on this proximal stud (5).

9. Vertebral osteosynthesis equipment according to claim 8, characterized in that said means comprise at least one flat surface (14) provided on the proximal stud (5) and at least one flat surface provided on the connecting part (3), whereas these flats surfaces are immediately close to one another when the connecting part (3) is engaged on the proximal stud (5).

10. Method for manufacturing the polyaxial anchoring member according to claim 1, characterized in that it comprises the steps consisting in:
providing, on the part intended for the proximal stud (5) of said polyaxial anchoring member, an articulation head (11) in the form of a spherical cap;
providing a cavity (16) in the proximal zone of the part intended for said body (6) of said anchoring member polyaxial, and, around this cavity (16), a wall (17) which may be crimped;
engaging said articulation head (11) into said cavity (16), and
crimping said wall (17) around said articulation head (11) so that this wall (17) exhibits a proximal form at least partially hemispherical.

11. Vertebral osteosynthesis equipment according to claim 2, characterized in that comprises at least one connecting part (3) exhibiting a rounded section (20) intended for surrounding a linking rod (2) and two parallel drilled wings (21), these wings (21) being intended for engaging onto said proximal stud (5) and for being clamped, using said clamping means (4), against a bearing surface (17) contained in said polyaxial anchoring member; the distal wing (21) of this connecting part (3) exhibits a distal cavity (28) in the form of a spherical cap, of greater diameter than that of said crimped wall (17), the connecting part (3) being intended for resting against this crimped wall (17) at of this cavity (28).

12. Vertebral osteosynthesis equipment according to claim 3, characterized in that comprises at least one connecting part (3) exhibiting a rounded section (20) intended for surrounding a linking rod (2) and two parallel drilled wings (21), these wings (21) being intended for engaging onto said proximal stud (5) and for being clamped, using said clamping means (4), against a bearing surface (17) contained in said polyaxial anchoring member; the distal wing (21) of this connecting part (3) exhibits a distal cavity (28) in the form of a spherical cap, of greater diameter than that of said crimped wall (17), the connecting part (3) being intended for resting against this crimped wall (17) at of this cavity (28).

13. Vertebral osteosynthesis equipment according to claim 2, characterized in that the stud (5) exhibits a zone (15) of reduced diameter, enabling to break its proximal portion after placing and clamping the nut (4).

14. Vertebral osteosynthesis equipment according to claim 3, characterized in that the stud (5) exhibits a zone (15) of reduced diameter, enabling to break its proximal portion after placing and clamping the nut (4).

15. Vertebral osteosynthesis equipment according to claim 4, characterized in that the stud (5) exhibits a zone (15) of reduced diameter, enabling to break its proximal portion after placing and clamping the nut (4).

16. Vertebral osteosynthesis equipment according to claim 2, characterized in that the proximal stud (5) and said connecting part (3) comprise means (14, 29) enabling to immobilise the proximal stud (5) in rotation when the connecting part (3) is engaged on this proximal stud (5).

17. Vertebral osteosynthesis equipment according to claim 3, characterized in that the proximal stud (5) and said connecting part (3) comprise means (14, 29) enabling to immobilise the proximal stud (5) in rotation when the connecting part (3) is engaged on this proximal stud (5).

18. Vertebral osteosynthesis equipment according to claim 4, characterized in that the proximal stud (5) and said connecting part (3) comprise means (14, 29) enabling to immobilise the proximal stud (5) in rotation when the connecting part (3) is engaged on this proximal stud (5).
